(19) United States
(12) Patent Application Publication
(10) Pub. No.: US 2022/0041233 A1
(43) Pub. Date: Feb. 10, 2022

(54) METHOD FOR PRODUCING A CUSTOM SADDLE SHELL, SADDLE SHELL, SADDLE AND BICYCLE WITH SADDLE

(71) Applicant: Horst HOHMUTH, Ulm (DE)

(72) Inventor: Horst HOHMUTH, Ulm (DE)

(21) Appl. No.: 17/429,227

(22) PCT Filed: Jan. 18, 2020
(86) PCT No.: PCT/DE2020/000005

§ 371 Date: Aug. 6, 2021

(30) Foreign Application Priority Data
Feb. 7, 2019 (DE) 10 2019 000 882.0

(51) Int. Cl. B62J 1/00; A61B 8/08

(52) U.S. Cl. CPC B62J 1/007; A61B 8/085; B62J 1/002

(57) ABSTRACT

The invention relates to a method having a plurality of steps for producing a customized saddle shell. Determining an individual extent of a genital region, in particular of a prostate. Producing the customized saddle shell having a recess for accommodating and relieving the genital region, in particular the prostate. The dimensions and/or geometry of the recess are/is determined as a function of the extent of the genital region, in particular of the prostate. Furthermore, the invention relates to the saddle shell, a saddle, and a bicycle having the saddle.

### TECHNICAL FIELD

The present invention relates to a saddle, a saddle shell, and a method for producing a customized saddle shell, as well as to a bicycle having said saddle.

### PRIOR ART

It is known that male cyclists increasingly experience erectile dysfunction caused by bicycle saddles and the associated pressure on the perineum and penile blood vessels. Especially men who suffer from enlarged prostates and who like to ride bicycles complain of numbness and reduced blood flow in the penis after long bicycle rides. This problem is not eliminated by the widely used gel saddles because the gel is displaced by the body weight. The man then presses down on a hard plastics shell of the saddle with his body weight, thereby compressing his prostate and the base of the penis.

A saddle having a narrow channel, designed to relieve the genital region, is known from EP 3 022 110 A1, for example. This channel is trapezoidal in a longitudinal axis of the saddle, has a constant width of 30 or 35 mm at the end pointing toward a rear edge of the saddle, extends across approximately three quarters of the saddle length, and tapers toward the nose of the saddle to a constant width of 10 or 20 mm. Narrow, long channels in a saddle generally result in the man's weight resting on the pubic bones with a relatively small contact area. This leads to a high pressure load on the pubic bones, which leads to chronic pain and inflammation of the periosteum (periostitis) of the sit and pubic bones (tuber ischiaticum and os pubis). Such saddle designs also can lead to tendinopathies of the piriformis muscle (“ham-string muscle”). These medical conditions are well known in sports medicine and result in a lengthy healing process. This condition can also be caused, among other things, by saddle designs which place high pressure loads on the periosteum.

The problem addressed by the present invention is therefore that of providing a saddle that is optimized with regard to a rider, an improved saddle shell and a method for producing a customized saddle shell.

### DISCLOSURE OF THE INVENTION

According to the invention, a method for producing a customized saddle shell is presented in claim 1. The method preferably comprises the following steps: determining an individual extent of a genital region, in particular of a prostate. producing the customized saddle shell having a recess for accommodating and relieving the genital region, in particular the prostate, wherein the dimension and/or geometry of the recess can be determined on the basis of the extent of the genital region, in particular of the prostate.

The term extent should preferably include the meaning of the size of a spatial dimension of an object. In particular, extent should include the volume, the spatial shape, density, weight or dimension such as height, length and width. The genital region includes in particular the penis, scrotum, testes, epididymis, spermatic ducts, prostate, and the tissue and vessels surrounding the aforementioned organs.

The production method according to the invention enables in particular men with enlarged prostates to ride bicycles with little discomfort or risk. The saddle shell manufactured in this way can alleviate numbness and reduced blood flow in the penis. A large saddle support surface can be achieved by means of the customized recess, which, in particular in contrast to known saddles with a large, elongate channel, distributes the weight of a rider sitting on the saddle shell more evenly and/or over a larger area and thus reduces a painful or even disease-causing pressure load, for example on the pubic bones of the rider. The saddle shell produced in this manner is able to prevent or reduce periostitis or tendinopathies of the piriformis muscle. Above all, the saddle shell produced in this manner can shift the weight of the rider sitting on the saddle shell onto his ischial tuberosities. The prostate region of the rider sitting on the saddle shell preferably comes to be positioned above the customized recess, as a result of which a region on or around the prostate, also called the prostate pressure point, can be relieved. In short, the method according to the invention can be used to produce a saddle shell which can achieve an improved pressure distribution for a rider sitting on said saddle shell while at the same time relieving the individual prostate of the rider. The customization is particularly advantageous because the size of the prostate or the extent of certain genital region can be different for different individuals. Once the prostate extent, dimension or volume has been determined, the recess can be adapted accordingly.

The extent is preferably determined by means of an imaging method comprising an ultrasound measurement method and in particular a transrectal ultrasound measurement. This transrectal ultrasound measurement can take place via the rectum, with measurements preferably being made at a distance of approximately one centimeter above a sphincter muscle. The method step of determining the extent does not serve to perform a diagnosis for any therapeutic purpose, but rather to determine data or physical quantities, such as the extent, the volume, the spatial shape, the density, the weight, or the dimension, such as height, length, and width, in order to be able to determine the dimension and/or geometry of the recess on the basis of the extent of the genital region, in particular of the prostate.

The shape of the prostate can depend on its volume and resemble an ellipsoid or a chestnut if the volume is less than 80 ml; the length of the prostate can be determined in a sagittal plane, and the width and height of the prostate can be determined in a transverse plane. As a result, its volume can be approximately determined using the following formula.

$$Volume = ( length \cdot width \cdot height ) \cdot \frac{π}{6}$$

For technical reasons, it may only be possible to determine the length of the prostate imprecisely. The prostate volume can approximately be determined from the width and the height in a transverse plane.

$$Volume = ( {width}^{2} \cdot height ) \cdot \frac{π}{6}$$

When the volume of the prostate is greater than 80 ml, its shape can resemble a sphere or a chestnut. Therefore, in particular, only the width in the transverse plane is used to determine the volume. Thus, the following formula is preferably used for determining the prostate volume:

$$Volume = ( {width}^{3} ) \cdot \frac{π}{6}$$

Values from the following table of values are preferred as dimensions for the recess for accommodating and relieving different prostate volumes:

| | Min. | Mean | Max. | First dimension | Second |
|---|---|---|---|---|---|
| Size | volume | volume | volume | of the recess in | dimension of the |
| range | in ml | in ml | in ml | mm | recess in mm |
| 1 | 0 | 10 | 20 | 20 | 25 |
| 2 | 20 | 30 | 40 | 25 | 30 |
| 3 | 40 | 50 | 60 | 30 | 35 |
| 4 | 60 | 75 | 90 | 35 | 40 |
| 5 | 90 | 105 | 120 | 40 | 45 |

Preferably, the first dimension is a length of the recess and the second dimension is a width of the recess. However, this allocation can also be advantageous in reverse. The production costs, for example tool costs, can be reduced if the various prostate volumes are combined in groups, and fixed dimensions of the recess are produced for these groups. In particular, a division into five size ranges is preferred.

The allocation of the prostate volumes, min. volume, mean volume and max. volume, to the five size ranges as well as to the dimensions is not conclusive, but can also be subdivided into finer size ranges, or be expanded upward or downward. A recess for a prostate volume greater than 120 ml is also possible, the dimension or dimensions of which can be determined by extrapolation or interpolation from the aforementioned table of values.

The aforementioned table of values advantageously takes into account a riding position of the rider sitting on the saddle shell, as well as a pressure distribution when taking into account the riding position of a cyclist inclined forward toward a handlebar. This can lead to a slight inclination of the pelvis forward, whereby pressure is increasingly exerted on the prostate pressure point, which is advantageously absorbed and relieved by means of the saddle shell.

In a preferred embodiment of the saddle shell, the shell can be produced by means of an injection molding process using a polymer such as polypropylene or acrylonitrile butadiene styrene copolymer (ABS for short). The division of the prostate volumes and, consequently, of the dimensions of the recess into a finite number of size ranges enables injection molding tools to be produced inexpensively.

Furthermore, according to the invention, a saddle shell according to claim 7 is provided. The saddle shell according to the invention preferably has a recess for accommodating and relieving pressure on a genital region, in particular the prostate, wherein the dimension and/or the geometry of the recess is formed according to the method according to the invention. The geometry includes in particular a circle, an ellipse, a chestnut shape, and an isosceles triangle.

Even more preferably, the recess is designed in the form of an isosceles Reuleaux triangle. The isosceles Reuleaux triangle has convex, i.e., outwardly curved, sides and can also have rounded corners. In order to make a comfortable sitting experience and improved injection molding possible, the recess can have beveled or rounded edges. A peripheral edge of the recess can be convex, i.e., outwardly curved. The recess takes into account in particular the anatomical design and size of the prostate and can reduce a damaging pressure response and the associated pain in the case of a rider sitting on it.

In an advantageous development of the saddle shell, the dimension and/or geometry comprises a length in a first axis, which preferably extends from the seat plane to the saddle nose or in a sagittal plane, and a width in a second axis of the saddle shell, which is preferably perpendicular to the first axis or extends in a transverse plane.

It is also considered advantageous that a region of the saddle shell is designed as a sitting surface and has a surface that is curved toward a seat plane and toward a saddle nose. The curved surface preferably comprises a saddle pad and is in particular formed from a plastics material, for example a thermoplastic elastomer such as polyurethane. In a further embodiment, the saddle pad can also be provided with a cover material.

In a further embodiment of the invention, a depression is formed along a longitudinal axis of the saddle shell. Therein, a center of the recess is preferably arranged at a predefined distance from a rear saddle edge in such a manner that it is positioned in the region of a perineum of a rider sitting on said saddle. This center can be understood as a fixed point for the dimensions and/or geometries. In the preferred embodiment, the predefined distance between the rear saddle edge and the fixed point of the recess is 80 to 100 mm, in particular 90 mm.

Furthermore, a saddle according to the invention according to claim 14 is proposed, which in particular has the saddle shell according to the invention, a frame and two saddle struts.

The saddle shell according to the invention is preferably glued to the frame. The saddle shell can be covered with a foam rubber layer to improve seating comfort.

In addition, a bicycle according to the invention having a saddle according to the invention is proposed. In principle, the saddle according to the invention can be any type of seat, chair, or seating device for any type of vehicle or furniture.

### BRIEF DESCRIPTION OF THE DRAWINGS

Embodiments of the invention are explained with reference to the drawings and to the description following them. In the drawings:

FIG. 1 shows a sectional view of a male genital region;

FIG. 2 shows a method according to an embodiment of the invention;

FIG. 3 shows a plan view of an upper side of a saddle shell according to a further embodiment of the invention;

FIG. 4 shows a perspective view of the top of the saddle shell according to a further embodiment of the invention;

FIG. 5 shows a plan view of a rear saddle edge of the saddle shell according to the invention;

FIG. 6A shows a diagram of a relationship between a first dimension of the recess 30 to the prostate volume 44 according to a further embodiment of the invention; and

FIG. 6B shows a diagram of a relationship between a second dimension of the recess 30 to the prostate volume 44 according to a further embodiment of the invention.

### EMBODIMENTS OF THE INVENTION

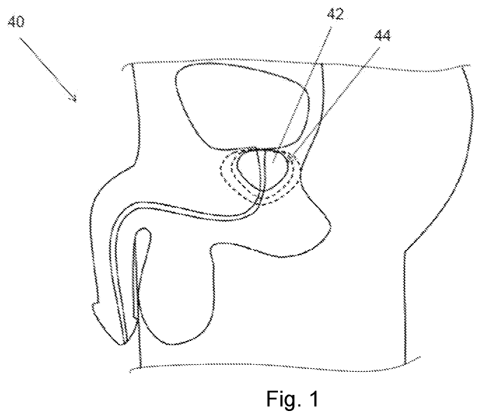

FIG. 1 shows a sectional view of a male genital region 40 as well as of a prostate 42 as a pyramidal, ellipsoidal, or chestnut-shaped organ, which is located below a urinary bladder in front of a rectum. A volume 44 of the prostate 42 varies, depending on age and condition, between slightly less than 20 ml up to 120 ml.

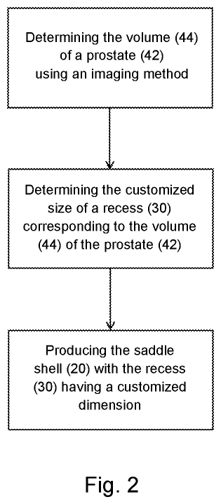

FIG. 2 shows steps of a method for producing a customized saddle shell 20 according to a first embodiment of the invention. Herein, the dimension and, based on this, the volume 44 of the prostate 42 are determined in a first step by means of a transrectal ultrasonic measurement. Then, based on the volume 44, the dimension of a recess 30 for accommodating and relieving the pressure on the prostate 42 is determined. The customized dimension of the recess having been determined in this manner, the customized saddle shell 20 is then produced by means of an injection molding process.

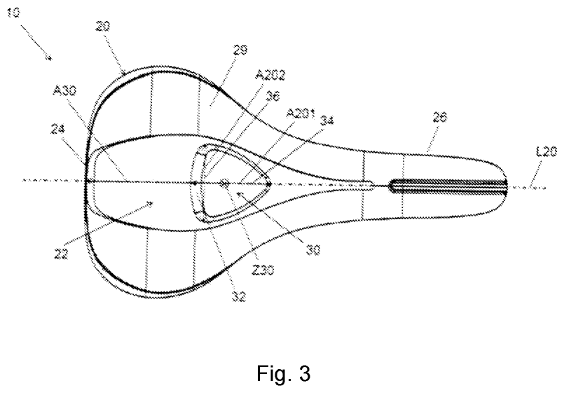

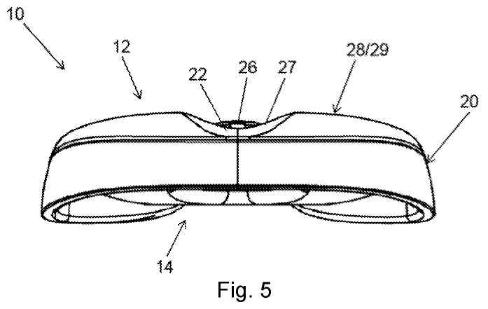

FIG. 3 shows a plan view of a saddle 10 which comprises a saddle shell 20. The saddle 10 has an upper side 12 and a lower side 14 opposite from it (see FIG. 5). Furthermore, the saddle 10 comprises a recess 30 designed to accommodate and relieve the genital region 40. A length 34 of the recess 30 extends in the direction of a first axis A201, and a width 36 extends in the direction of a second axis A202 of the saddle shell 20. The recess 30 penetrates the saddle shell 20 and is designed as an isosceles triangle. A peripheral edge 32 of the recess 30 has a convex, i.e., outwardly curved, shape. This allows the anatomical manifestation and size of the prostate 42 to be taken into account and thus prevents a damaging pressure response and associated pain in the genital region 40 of a rider sitting on the saddle 10.

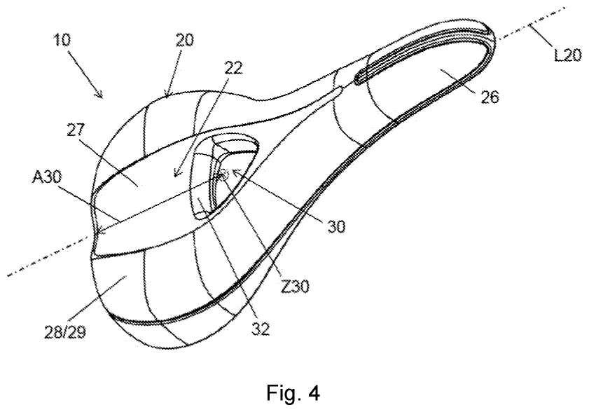

Furthermore, FIG. 3 shows a region of the saddle shell 20 designed as a sitting surface and a surface 27 curved toward a seat plane 28 (see FIG. 5) and toward a saddle nose 26 (see FIG. 4). Therein, the curved surface 27 forms a saddle pad 29. The saddle pad 29 is preferably formed from a thermoplastic elastomer such as polyurethane. The saddle pad 29 can also be provided with a cover material. The shape of the saddle pad 29 is designed in such a way that the prostate 42 is relieved in the perineum region and the pressure, resulting from the body weight of the rider in question, is thus transferred to the ischial tuberosities.

In the saddle pad 29 itself, a depression 22, which tapers toward the saddle nose 26, is formed along a longitudinal axis L20. A center Z30 of the recess 30 is formed in this depression 22 at a predefined distance A30 from a rear saddle edge 24. The distance A30 between the rear saddle edge 24 and the center Z30 of the recess 30 is 90 mm. The geometry based on the position of the center Z30 and the length 34 and width 36 of the recess 30 is selected as an isosceles triangle or chestnut shape, depending on the anatomical conditions of the prostate 42 when sitting on the saddle 10.

The perspective view of the upper side 12 of the saddle shell 20 in FIG. 4 shows the depression 22 which is described in FIG. 3 and in which the recess 30 is formed, and the surface 27, which is designed to be curved toward the seat plane 28 and toward the saddle nose 26.

Furthermore, the plan view of the rear saddle edge 24 of the saddle shell 20 shown in FIG. 5 illustrates the depression 22 formed in the saddle pad 29, as well as the curved surface 27 extending toward the seat plane 28 and toward the saddle nose 26.

The saddle 10 having the saddle shell 20 furthermore has at least two saddle struts (not shown in the figures), which are preferably formed from a steel alloy with high tensile strength, such as chromium-molybdenum steel.

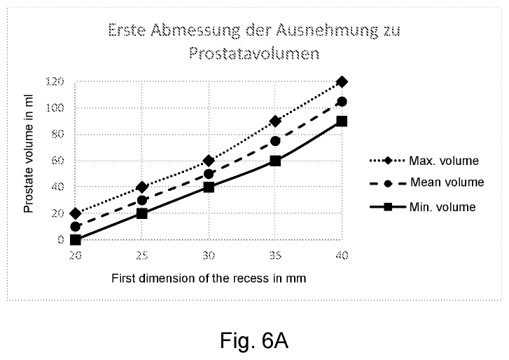

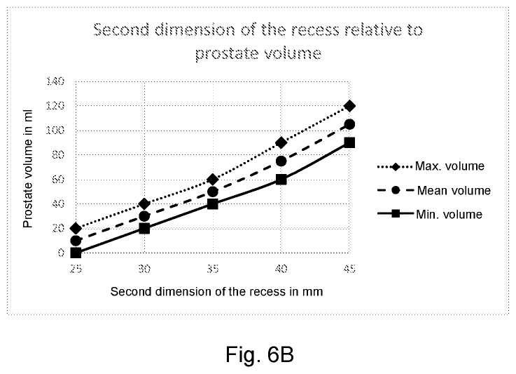

FIG. 6A shows a diagram which illustrates a relationship between a first dimension of the recess 30 and the prostate volume 44. FIG. 6B shows a diagram which illustrates a relationship between a second dimension of the recess 30 and the prostate volume 44. The diagrams in FIG. 6A, 6B each show graphs for volume or size ranges, namely max. volume, mean volume and min. volume. The first dimension corresponds to the length 34 and the second dimension corresponds to the width 36. According to a further embodiment, the first dimension corresponds to the width 36 and the second dimension corresponds to the length 34.

### LIST OF REFERENCE SIGNS

10 Saddle
12 Upper side
14 Lower side
20 Saddle shell
22 Depression
24 Saddle edge
26 Saddle nose
27 Curved surface
28 Seat plane/sitting surface
29 Saddle pad
30 Recess
32 Edge
34 Length (of 30)
36 Width (of 30)
40 Genital region
42 Prostate
44 Volume (of 42)

A30 Distance (between 24 and 30)
A201 (First) axis (of 20)
A202 (Second) axis (of 20)
L20 Longitudinal axis (of 20)
Z30 Center (of 30)

1-15. (canceled)

16. A method for producing a customized saddle shell, comprising the following steps:
determining an individual extent of a genital region, in particular of a prostate, and producing the customized saddle shell having a recess for accommodating and relieving the genital region, in particular the prostate, wherein the dimension and/or geometry of the recess is determined on the basis of the prostate.

17. The method according to claim 16, wherein the extent is determined by means of an imaging method.

18. The method according to claim 17, wherein the imaging method used to determine the extent comprises an ultrasound measuring method.

19. The method according to claim 16, wherein the dimension of the recess is between 20 and 45 mm.

20. The method according to claim 16, wherein the dimension of the recess is determined from one of the following five size ranges:
a first size range for prostate volumes of less than 20 ml corresponds to a first recess dimension of 20 mm and/or a second recess dimension of 25 mm,
a second size range for prostate volumes from 20 ml to 40 ml corresponds to a first recess dimension of 25 mm and/or a second recess dimension of 30 mm,
a third size range for prostate volumes from 40 ml to 60 ml corresponds to a first recess dimension of 30 mm and/or a second recess dimension of 35 mm,
a fourth size range for prostate volumes of 60 ml to 90 ml corresponds to a first recess dimension of 40 mm and/or a second recess dimension of 45 mm, and
a fifth size range for prostate volumes from 90 ml to 120 ml corresponds to a first recess dimension of 40 mm and/or a second recess dimension of 45 mm.

21. The method according to claim 16, wherein the saddle shell is produced by means of an injection molding process.

22. A saddle shell, having a recess for accommodating and relieving a genital region, in particular the prostate, wherein the dimension and/or the geometry of the recess is formed according to the method in claim 16.

23. The saddle shell according to claim 22, wherein the recess penetrates the saddle shell and is designed as an isosceles Reuleaux triangle.

24. The saddle shell according to claim 22, wherein a peripheral edge of the recess has a convex shape.

25. The saddle shell according to claim 22, wherein the dimension and/or geometry comprises a length in a first axis (A201) and a width in a second axis (A202) of the saddle shell.

26. The saddle shell according to claim 16, wherein a region of the saddle shell (20) is designed as a sitting surface and has a surface curved toward a seat plane and toward a saddle nose.

27. The saddle shell according to claim 26, wherein the curved surface comprises a saddle pad, the saddle pad being formed from a plastics material.

28. The saddle shell according to claim 27, wherein a depression is formed in the saddle pad along a longitudinal axis (L20), in which a center (Z30) of the recess is arranged at a predefined distance (A30) from a rear saddle edge.

29. The saddle having a saddle shell according to claim 22, and having a frame and two saddle struts.

30. A bicycle having a saddle according to claim 29.
